(19) United States
(12) Patent Application Publication
(10) Pub. No.: US 2025/0289775 A1
(43) Pub. Date: Sep. 18, 2025

(54) SUPPORTED CATALYST AND PREPARATION METHOD THEREOF, AND METHOD FOR PREPARING HIGH-CARBON KETONE

(71) Applicant: ZHEJIANG SAINON CHEMICAL CO., LTD, Zhejiang (CN)

(72) Inventors: Jianhui LI, Zhejiang (CN); Shuqun Xu, Zhejiang (CN); Weipeng Li, Zhejiang (CN); Lizhong Zhang, Zhejiang (CN); Ji'an Fan, Zhejiang (CN)

(73) Assignee: ZHEJIANG SAINON CHEMICAL CO., LTD, Zhejiang (CN)

(21) Appl. No.: 18/957,105

(22) Filed: Nov. 22, 2024

(30) Foreign Application Priority Data
Mar. 15, 2024 (CN) 202410296354.9

(51) Int. Cl. C07C 45/45; B01J 23/755; B01J 23/80; B01J 23/889; B01J 31/06; B01J 37/04; B01J 37/10; C07C 45/71

(52) U.S. Cl. CPC C07C 45/45; B01J 23/755; B01J 23/80; B01J 23/8892; B01J 31/06; B01J 37/04; B01J 37/10; C07C 45/71

(57) ABSTRACT

A supported catalyst and a preparation method thereof, and a preparation method of a high-carbon ketone are provided. The method for preparing the supported catalyst includes: mixing a transition metal nitrate and/or a transition metal acetate as a reaction substrate, a polyolefin powder porous material as a catalyst carrier, and water as a reaction medium evenly to obtain a mixture, and subjecting the mixture to drying and calcination in sequence to obtain the supported catalyst.

### CROSS REFERENCE TO RELATED APPLICATION

This patent application claims the benefit and priority of Chinese Patent Application No. 202410296354.9 filed with the China National Intellectual Property Administration on Mar. 15, 2024, the disclosure of which is incorporated by reference herein in its entirety as part of the present application.

### TECHNICAL FIELD

The present disclosure belongs to the technical field of chemical catalysis, and in particular relates to a supported catalyst and a preparation method thereof, and a method for preparing a high-carbon ketone.

### BACKGROUND

It is known that the size of ketone molecules would affect reactivity and energy release during the reaction. Compared with small-molecule ketones, high-carbon ketones have relatively large molecular structures and could achieve more complex and diverse chemical reactions. In industrial production, the high-carbon ketones could be used as an excellent organic solvent, as well as an important organic chemical raw material that is widely used in the synthesis of fragrances, organic synthesis, and synthesis of pharmaceutical intermediates. However, the existing methods for synthesizing high-carbon ketones are limited, and generally have a complicated reaction process and many by-products. For example, high-carbon ketones could be prepared through oxidative dehydrogenation of the corresponding alcohols, or through aldol condensation of ketones and aldehydes followed by hydrogenation. However, these methods have the disadvantages of high production costs and generally low product selectivity. In addition, a method for preparing pentanone by condensation of acetaldehyde and acetone, dehydration, and hydrogenation in the presence of hydrogen has been reported in the prior art. However, a target product obtained by the method has complex components and low selectivity, and the method requires high-pressure hydrogen to participate in the reactions, thus placing high demands on the reaction conditions.

In addition, it is also disclosed in the prior art that α-H-containing ketones and other active α-H-containing carbonyl compounds, such as ketones and aldehydes small-molecules, are subjected to aldol condensation to obtain condensation coupling products, and then the condensation coupling products are subjected to dehydration and hydrogenation to obtain alkyl-grafted high-carbon ketones. There are also methods of using alcohols as a condensation coupling reagent to couple with the α-H-containing ketones to prepare high-carbon ketones. However, the synthesis of high-carbon ketones above requires four steps of hydrogen borrowing (alcohol dehydrogenation to obtain small-molecule carbonyl compounds containing active α-H), aldol condensation (the substrate containing α-H ketones), dehydration, and hydrogenation, which puts forward extremely high requirements on the design of catalysts. The catalyst systems reported in the prior art include supported noble metal catalysts, homogeneous catalysts, or composite oxide catalysts. For example, Chinese patent CN106732555A reports a Pd/C catalyst for the α-alkylation of ketones and alcohols. However, a catalytic reaction of acetophenone and n-butanol requires a solvent 1,4-dioxane, which easily forms explosive peroxides, requires precise process control during production, and has low safety. For another example, Chinese patent CN111889105A discloses a bifunctional catalyst for preparing 3-pentanone by alkylation of methanol and butanone, where the bifunctional catalyst includes 2% to 30% of nickel oxide, 40% to 90% of magnesium oxide, 10% to 30% of aluminum oxide, and 0% to 20% of zinc oxide. The raw materials methanol and butanone undergo a reaction at 220° C. to 350° C. in a molar ratio of (5-15):1. However, the reaction has high temperature, great control difficulty, and poor yield of 3-pentanone. For example, Chinese patents CN110423190A and CN106905125A report an iron complex and a cobalt complex as catalysts in catalyzing α-alkylation of ketones, respectively. The catalysts are prepared by reacting 4′-dimethylaminophenyl-2,2′:6′,2″-terpyridine with anhydrous ferrous chloride, and 2,2:6,2″-terpyridine with cobalt chloride, respectively. However, the above patents generally have problems such as expensive catalysts, complicated and harsh reaction conditions, and difficulty in product separation and catalyst recovery.

In view of this, the present disclosure is specifically proposed.

### SUMMARY

In view of the above-mentioned existing technical problems, the present disclosure is to provide a supported catalyst and a preparation method thereof, and a method for preparing a high-carbon ketone.

The present disclosure provides a method for preparing a supported catalyst, including:
mixing a transition metal nitrate and/or a transition metal acetate as a reaction substrate, a polyolefin powder porous material as a catalyst carrier, and water as a reaction medium evenly to obtain a mixture, and subjecting the mixture to drying and calcination in sequence to obtain the supported catalyst.

In some embodiments, a transition metal in the transition metal nitrate and/or the transition metal acetate is an element selected from the group consisting of Group VIIB, Group VIII, Group IB, and Group IIB in periodic table of elements, and the transition metal is a non-precious metal element.

In some embodiments, the transition metal is selected from a transition metal element in a fourth period of the periodic table of elements.

In some embodiments, the method for preparing the supported catalyst includes the following steps:
S1, dissolving the transition metal nitrate and/or the transition metal acetate in an appropriate amount of the water to obtain a solution;
S2, adding the polyolefin powder porous material into the solution obtained in step S1 and mixing evenly by stirring at ambient temperature to obtain a mixed system;
S3, placing the mixed system obtained in step S2 in an oven, and drying the mixed system at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

In some embodiments, the heating is conducted at a heating rate of 2° C./min to 10° C./min, and the calcination temperature is in a range of 750° C. to 850° C.

The present disclosure further provides a supported catalyst prepared by the method for preparing the supported catalyst described above.

In some embodiments, the supported catalyst is any one or more selected from the group consisting of Ni5-Cu1/Polypropylene(PP), Ni5/PP, Ni2-Cu1/PP, Ni5-Co1/PP, Ni5-Fe1/PP, Co5-Zn1/PP, Mn5-Cu1/PP, Ni5-Cu1/polyethylene(PE), and Ni5-Cu1/PE.

The present disclosure further provides a method for preparing a high-carbon ketone, including:
subjecting an alcohol and an α-H-containing ketone that serve as reaction substrates to condensation coupling reaction in the presence of the supported catalyst as a reaction catalyst in a closed reactor at a reaction temperature of 120° C. to 250° C. under an initial pressure of atmospheric pressure to obtain the high-carbon ketone.

In some embodiments, the high-carbon ketone refers to ketones with a carbon chain length of 5-20.

In some embodiments, the alcohol is one or more selected from the group consisting of an aliphatic alcohol, an aromatic alcohol, an alicyclic alcohol, and an alcohol containing other heteroatom substituents; and the α-H-containing ketone is one or more selected from the group consisting of an aliphatic ketone, an aromatic ketone, an alicyclic ketone, and a ketone containing other heteroatom substituents.

In some embodiments, in the reaction substrates for preparing high-carbon ketone, a molar ratio of the α-H-containing ketone to the alcohol is in a range of 1:2 to 2:1; and a feeding ratio of the α-H-containing ketone to the supported catalyst is 0.2 g to 0.3 g of the supported catalyst per 1 mol of the α-H-containing ketone. The condensation coupling is conducted at a temperature of 160° C. to 210° C. for 30 min to 300 min.

The embodiments of present disclosure have the following beneficial effects:

In the present disclosure, the supported catalyst prepared through in-situ reduction can be directly used for the condensation coupling for synthesizing high-carbon ketone without reduction of the supported catalyst, thereby simplifying the catalyst preparation and reducing production costs.

On this basis, the supported catalyst according to the present disclosure does not contain precious metals, has low cost, and is easy to be manufactured.

In addition, the supported catalyst according to the present disclosure can be directly used to catalyze the condensation coupling of α-H-containing ketone and small-molecule alcohol, and the target product high-carbon ketone can be obtained with a high selectivity. Moreover, no external solvent and hydrogen source are required during the reaction. The preparation method has simple operations, a low cost, and a simple, efficient, and easy-to-control reaction process, as well as high safety, few by-products and high product selectivity.

### DETAILED DESCRIPTION OF THE EMBODIMENTS

The technical solutions of the present disclosure will be clearly and completely described below with reference to the embodiments. Apparently, the described embodiments are merely some rather than all of the embodiments of the present disclosure. All other embodiments obtained by those skilled in the art based on the described embodiments of the application shall fall within the scope of the application.

It should be understood that the term “and/or” in this specification merely describes associations between associated objects, and it indicates three types of relationships. For example, A and/or B may indicate that A exists alone, A and B coexist, or B exists alone. In addition, the character “/” in this specification generally indicates that the associated objects are in an “or” relationship.

In the description of this specification, it should be understood that the terms such as “substantially”, “approximate to”, “approximately”, “about”, “roughly”, and “in general” described in the claims and embodiments of the present disclosure mean general agreement within a reasonable process operation range or tolerance range, rather than an exact value.

It should be noted that terms “including”, “comprising”, or any other variants thereof in the present disclosure are intended to cover non-exclusive inclusion such that a process, method, article, or apparatus including a series of elements includes not only those elements but also other elements not explicitly listed, or elements inherent to such a process, method, article, or apparatus. Without further limitation, an element qualified by the phrase “including a . . . ” does not exclude the presence of an additional identical element in the process, method, article, or apparatus including the element. In addition, it should be noted that the scope of the methods and devices in the embodiments of the present disclosure is not limited to conducting functions in the order shown or discussed, and may also include conducting functions in a substantially simultaneous manner or in reverse order depending on the functions involved. For example, the described methods may be conducted in an order different from that described, and various steps may be added, omitted, or combined. Additionally, features described with reference to certain examples may be combined in other examples.

The present disclosure provides a method for preparing a supported catalyst, including:
mixing a transition metal nitrate and/or a transition metal acetate as a reaction substrate, a polyolefin powder porous material as a catalyst carrier, and water as a reaction medium evenly to obtain a mixture, and subjecting the mixture to drying and calcination in sequence to obtain the supported catalyst.

In some embodiments, a transition metal in the transition metal nitrate and/or the transition metal acetate is an element selected from the group consisting of Group VIIB, Group VIII, Group IB, and Group IIB in periodic table of elements, and the transition metal is a non-precious metal element.

In some embodiments, the transition metal is selected from a transition metal element in a fourth period of the periodic table of elements.

As some embodiments of the present disclosure, the transition metal in the transition metal nitrate and/or the transition metal acetate is one or more selected from the group consisting of Mn, Ni, Co, Fe, Zn, and Cu. In some embodiments, the transition metal is one or more selected from the group consisting of Ni, Co, and Cu.

As some embodiments of the present disclosure, the polyolefin powder porous material is one or more selected from the group consisting of polyethylene (PE) and polypropylene (PP) powder porous materials. In some embodiments, the polyolefin powder porous material is the PP powder porous material.

In some embodiments, the method for preparing the supported catalyst includes the following steps:
S1, dissolving the transition metal nitrate and/or the transition metal acetate in an appropriate amount of the water to obtain a solution;
S2, adding the polyolefin powder porous material into the solution obtained in step S1 and mixing evenly by stirring at ambient temperature to obtain a mixed system;
S3, subjecting the mixed system obtained in step S2 to standing for 2 h to 10 h, placing a resulting system in an oven, and drying at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

As some embodiments of the present disclosure, in step S1, a weight ratio of the water to the transition metal nitrate and/or transition metal acetate is in a range of 100:0.1 to 100:50.

In some embodiments, in step S2, a weight ratio of the transition metal nitrate and/or transition metal acetate to the polyolefin powder porous material is in a range of 3:100 to 50:100.

In some embodiments, in step S4, the heating is conducted at a heating rate of 2° C./min to 10° C./min, and the calcination temperature is in a range of 750° C. to 850° C.

In some embodiments, the method for preparing the supported catalyst includes the following steps:
S1, dissolving the transition metal nitrate and/or the transition metal acetate in an appropriate amount of the water to obtain a solution;
S2a, adding the polyolefin powder porous material into the solution obtained in step S1 and mixing evenly by stirring at ambient temperature to obtain a mixed system;
S2b, dispersing an appropriate amount of an inorganic porous material in an organic solvent, stirring and mixing evenly to obtain a mixed material; pressurizing the mixed material to 0.5 MPa to 3 MPa and maintaining for 3 min to 10 min, and then reducing a pressure of the mixed material to atmospheric pressure at a speed of 0.1 MPa/min to 0.3 MPa/min; and filtering a resulting material to obtain a pretreated inorganic porous material;
S2c, adding the pretreated inorganic porous material into the mixed system obtained in step S2a, and mixing evenly by stirring to obtain a mixture system;
S3, subjecting the mixture system obtained in step S2c to standing for 2 h to 10 h and drying in an oven at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

As some embodiments of the present disclosure, the inorganic porous material is one or more selected from the group consisting of porous ceramic materials, porous molecular sieves, porous activated carbon, and natural inorganic porous materials such as natural zeolite and diatomaceous earth. In some embodiments, the inorganic porous material is the porous ceramic material.

In some embodiments, the filtering in step S2b is normal-pressure filtering.

In some embodiments, the polyolefin powder porous material has a particle size of less than or equal to 1 μm.

In some embodiments, the inorganic porous material has a particle size of less than or equal to 10 mm, and preferably, the inorganic porous material has a particle size of less than or equal to 1 mm.

As an embodiment of the present disclosure, the pore size of the inorganic porous material is less than or equal to 1.2 times the particle size of the polyolefin powder porous material.

In some embodiments, in step S2b, a weight ratio of the inorganic porous material to the polyolefin powder porous material is in a range of 3:1 to 5:1.

In some embodiments, in step S2b, a weight ratio of the inorganic porous material to the organic solvent is in a range of 0.1:1 to 0.3:1.

As some embodiments of the present disclosure, in step S2b, the organic solvent is a non-polar organic solvent being one or more selected from the group consisting of cyclohexane and benzene.

In the process of preparing the supported catalyst, a carrier of the polyolefin powder porous material is formed by the inorganic porous material. On this basis, by pre-filling the inorganic porous material with an organic solvent that is immiscible with water, the organic solvent can be used to preemptively fill and occupy the pores inside the inorganic porous material. When the inorganic porous material is mixed with the mixed system obtained in step S2a, the polyolefin powder porous material therein can preferentially adhere to and combine with a surface of the inorganic porous material, maintaining the high activity of a catalyst polymer formed after the polyolefin powder porous material adheres to the surface of the inorganic porous material. At the same time, by using large-particle inorganic porous materials, efficient and convenient recovery of the supported catalyst is achieved.

In addition, the present disclosure further provides a supported catalyst for preparing a high-carbon ketone, where the supported catalyst is prepared by the method described above.

As some embodiments of the present disclosure, the supported catalyst is any one selected from the group consisting of Ni5-Cu1/PP, Ni5/PP, Ni2-Cu1/PP, Ni5-Co1/PP, Ni5-Fe1/PP, Co5-Zn1/PP, Mn5-Cu1/PP, Ni5-Cu1/PE, and Ni5-Cu1/PE.

In the present disclosure, the catalyst prepared by calcination has a fluffy porous structure and a large specific surface area, shows the characteristics of highly dispersed active components and rich oxygen vacancies, and can catalyze condensation coupling efficiently and stably.

Furthermore, the present disclosure further provides a method for preparing a high-carbon ketone, including:
subjecting an alcohol and an α-H-containing ketone that serve as reaction substrates to condensation coupling in the presence of the supported catalyst as described above as a reaction catalyst in a closed reactor at a reaction temperature of 120° C. to 250° C. under an initial pressure of atmospheric pressure to obtain the high-carbon ketone.

In some embodiments, the alcohol is one or more selected from the group consisting of an aliphatic alcohol, an aromatic alcohol, an alicyclic alcohol, and an alcohol containing other heteroatom substituents.

In some embodiments, the alcohol is one or more selected from the group consisting of ethanol, n-propanol, isopropanol, ethylene glycol, phenylethanol, cyclohexanol, and ethanolamine.

In some embodiments, the α-H-containing ketone is one or more selected from the group consisting of an aliphatic ketone, an aromatic ketone, an alicyclic ketone, and a ketone containing other heteroatom substituents.

In some embodiments, the α-H-containing ketone is one or more selected from the group consisting of acetone, butanone, pentanone, acetophenone, cyclohexanone, and 1-amino-2-propanone.

In some embodiments, a molar ratio of the α-H-containing ketone to the alcohol is in a range of 1:2 to 2:1; and a feeding ratio of the α-H-containing ketone to the supported catalyst is 0.2 g to 0.3 g of the supported catalyst per 1 mol of the α-H-containing ketone. The condensation coupling is conducted at a temperature of 160° C. to 210° C. for 30 min to 300 min.

In the present disclosure, the polyolefin powder is used as a catalyst carrier, which could reduce the transition metal in situ during the calcination decomposition. The catalyst obtained after cooling can be directly used for the condensation coupling of α-H-containing ketone and alcohol to obtain the high-carbon ketone with a high selectivity without an independent reduction step. Moreover, the catalyst is cheap and easy to be obtained. When the catalyst prepared above is used in the condensation coupling of α-H-containing ketone and alcohol, the reaction does not require an external solvent or high-pressure hydrogen. The reaction is easy to be implemented, the catalyst raw materials used are easy to be obtained, and the preparation method is simple. In addition, a catalytic conversion rate for condensation coupling is high and the applicable alcohol categories are wide. The abundant small-molecule alcohol and α-H-containing ketone can be efficiently reacted to obtain the high-carbon ketone, with conversion rates of the alcohol and ketone of not less than 80% respectively, and a selectivity of high-carbon ketone of not less than 90%. In addition, the preparation method can operate stably for a long time and has desirable industrial application prospects.

The present disclosure will be further described in detail below with reference to the examples, but the present disclosure is not limited to the content of the examples.

### Example 1

### Preparation of a Supported Catalyst:

A certain amount of nickel nitrate and copper nitrate were fully dissolved in an appropriate amount of water at ambient temperature, and then a PP porous powder material was added thereto and mixed evenly. After standing for 4 h, a resulting mixture was placed and dried in a muffle furnace at 100° C. A resulting dried material was placed in a calcination device, heated and calcinated at 800° C. for 4 h, where the dried material was heated at a heating rate of 9° C./min. After the calcination was completed, a resulting material was cooled to ambient temperature with the furnace to obtain a target catalyst, recorded as Ni5-Cu1/PP.

### Examples 2-7

### Preparation of Supported Catalysts:

Examples 2-7 were conducted according to the method of Example 1, except that the kind of the transition metal nitrate, the ratio of the transition metal nitrate, the calcination temperature, the heating rate, and the calcination time were adjusted. The calcination temperatures were set to 800° C., 850° C., 900° C., 750° C., 800° C., and 750° C., respectively. The heating rates were set to 7° C./min, 8° C./min, 9° C./min, 10° C./min, 6° C./min, and 4° C./min, respectively. The calcination time was set to 2 h, 3 h, 5 h, 6 h, 7 h, and 8 h, respectively. The catalysts obtained finally were recorded as: Ni5/PP, Ni2-Cu1/PP, Ni5-Co1/PP, Ni5-Fe1/PP, Co5-Zn1/PP, and Mn5-Cu1/PP, respectively.

### Example 8

### Preparation of a Supported Catalyst:

A certain amount of nickel acetate and copper nitrate were fully dissolved in an appropriate amount of water at ambient temperature, and then a PE porous powder material was added thereto and mixed evenly. After standing for 2 h, a resulting mixture was placed and dried in a muffle furnace at 80° C. A resulting dried material was placed in a calcination device, heated and calcinated at 700° C. for 4 h, where the dried material was heated at a heating rate of 2° C./min. After the calcination was completed, a resulting material was cooled to ambient temperature with the furnace to obtain a target catalyst, recorded as Ni5-Cu1/PE.

### Example 9

### Preparation of a Supported Catalyst:

A certain amount of nickel nitrate and copper nitrate were fully dissolved in an appropriate amount of water at ambient temperature, and then a PE porous powder material was added thereto and mixed evenly. After standing for 2 h, a resulting mixture was placed and dried in a muffle furnace at 80° C. A resulting dried material was placed in a calcination device, heated and calcinated at 700° C. for 4 h, where the dried material was heated at a heating rate of 2° C./min. After the calcination was completed, a resulting material was cooled to ambient temperature with the furnace to obtain a target catalyst, recorded as Ni5-Cu1/PE.

### Example 10

### Preparation of a Supported Catalyst:

A certain amount of nickel nitrate and copper nitrate were fully dissolved in an appropriate amount of water at ambient temperature, and then a PE porous powder material was added thereto and mixed evenly to obtain a mixed system. An appropriate amount of ceramic porous material was dispersed in an organic solvent, stirred and mixed evenly, and then pressurized to 1 MPa and maintained for 5 min, and reduced to an atmospheric pressure at 0.2 MPa/min, then, a resulting material was filtered to obtain a pretreated ceramic porous material. The pretreated ceramic porous material was added into the mixed system of nickel nitrate, copper nitrate, and PE porous powder material, stirred until evenly mixed to obtain a mixture. The mixture was subjected to standing for 5 h, and then placed and dried in an oven at 90° C. to a constant weight. A resulting dried material was placed in a calcination device, heated and calcinated at 700° C. for 4 h, where the dried material was heated at a heating rate of 2° C./min. After the calcination was completed, a resulting material was cooled to ambient temperature with the furnace to obtain a target catalyst, recorded as Ni5-Cu1/PE/ceramic.

The substrate feeding conditions during the preparation of the catalysts in Examples 1 to 10 were shown in Table 1:

TABLE 1
Substrate feeding during preparation of catalysts

| Examples for | Transition metal nitrate/Transition | | | |
|---|---|---|---|---|
| catalyst | metal acetate | | Catalyst | Catalyst |
| preparation | Type | ratio | carrier | obtained |
| Example 1 | Nickel nitrate, copper nitrate | 5:1 | PP | Ni5—Cu1/PP |
| Example 2 | Nickel nitrate | — | PP | Ni5/PP |
| Example 3 | Nickel nitrate, copper nitrate | 2:1 | PP | Ni2—Cu1/PP |
| Example 4 | Nickel nitrate, cobalt nitrate | 5:1 | PP | Ni5—Co1/PP |
| Example 5 | Nickel nitrate, ferric nitrate | 5:1 | PP | Ni5—Fe1/PP |
| Example 6 | Cobalt nitrate, zinc nitrate | 5:1 | PP | Co5—Zn1/PP |
| Example 7 | Manganese nitrate, copper | 5:1 | PP | Mn5—Cu1/PP |
| | nitrate | | | |
| Example 8 | Nickel acetate, copper nitrate | 5:1 | PE | Ni5—Cu1/PE |
| Example 9 | Nickel nitrate, copper nitrate | 5:1 | PE | Ni5—Cu1/PE |
| Example 10 | Nickel nitrate, copper nitrate | 5:1 | PE | Ni5—Cu1/PE/ceramic |

In the supported catalysts of the present disclosure, polyolefin powder was selected as a catalyst carrier, having a large specific surface area, high porosity, and uniform pore size distribution, which could increase a contact area between the catalyst and the reactant, thereby increasing a reaction rate. At the same time, the polyolefin powder shows excellent mechanical properties and corrosion resistance. When the post-transition metal loaded by the polyolefin powder is used as a catalyst for the ketone-alcohol condensation coupling, the catalyst has a stable catalytic performance, and could operate stably for a long time, which shows desirable industrial application prospects.

More importantly, the polyolefin powder is used as a catalyst carrier, which could reduce the transition metal in situ during the calcination decomposition. The catalyst obtained after cooling could be directly used for the condensation coupling of α-H-containing ketone and alcohol to obtain the high-carbon ketone with a high selectivity without an independent reduction step. Moreover, the catalyst is cheap and easy to be obtained.

### Example 11

### Preparation of a High-Carbon Ketone:

0.05 g of the catalyst Ni5-Cu1/PP, 20 mL of ethanol, and 20 mL of acetone were added into a 100 mL high-pressure reactor in sequence, and a reaction was conducted at 175° C. for 5 h under stirring at 500 r/min. The results of gas chromatography analysis show that the reaction results in a conversion rate of ethanol of 89%, a conversion rate of acetone of 84%, and a selectivity of 2-pentanone of 90%.

### Examples 12-30

Examples 12-30 were conducted according to the method of Example 11, except that the reaction conditions and substrates were adjusted. The composition of the product after reaction was analyzed. The reaction conditions and catalytic performance results of condensation coupling examples are shown in Table 2.

TABLE 2
Reaction conditions and catalytic performance results of condensation coupling examples

| | | | | | | A | A | A |
|---|---|---|---|---|---|---|---|---|
| | | | | | | conversion | conversion | selectivity |
| | | | | | | rate of | rate of | of corresponding |
| | | | | | | substrate | substrate | high-carbon |
| | | Temperature, | Time, | Substrate | Substrate | ketone, | alcohol, | ketone, |
| Examples | Catalyst | ° C. | h | ketone | alcohol | % | % | % |
| 11 | Ni5-Cu1/PP | 175 | 5 | Acetone | Ethanol | 84 | 89 | 90 |
| 12 | Ni5-Cu1/PP | 185 | 1 | Acetone | Ethanol | 83 | 84 | 91 |
| 13 | Ni5-Cu1/PP | 195 | 0.5 | Acetone | Ethanol | 85 | 88 | 93 |
| 14 | Ni5-Cu1/PP | 200 | 0.5 | Acetone | n-propanol | 82 | 82 | 92 |
| 15 | Ni5-Cul/PP | 190 | 1.5 | Acetone | Ethanol | 88 | 90 | 93 |
| 16 | Ni2-Cu1/PP | 200 | 2 | Acetone | Isopropanol | 86 | 83 | 93 |
| 17 | Ni5-Co1/PP | 210 | 2.5 | Acetone | Ethylene | 82 | 84 | 92 |
| | | | | | glycol | | | |
| 18 | Ni5/PP | 220 | 3 | Acetone | Glycerol | 81 | 86 | 90 |
| 19 | Ni5-Fe1/PP | 175 | 3.5 | Butanone | Ethanol | 88 | 82 | 91 |
| 20 | Co5-Zn1/PP | 130 | 4 | 2-Pentanone | Ethanol | 80 | 80 | 90 |
| 21 | Mn5-Cu1/PP | 175 | 4.5 | Butanone | Ethanol | 86 | 89 | 92 |
| 22 | Ni5-Cu1/PP | 175 | 5 | Acetophenone | Ethanol | 82 | 83 | 91 |
| 23 | Ni5-Cu1/PP | 120 | 3 | Cyclohexanone | Ethanol | 85 | 89 | 90 |
| 24 | Ni5-Cu1/PP | 240 | 4 | 1-amino-2- | Ethanol | 81 | 88 | 94 |
| | | | | propanone | | | | |
| 25 | Ni5-Cu1/PP | 160 | 1 | Acetone | Phenethyl | 86 | 90 | 91 |
| | | | | | alcohol | | | |
| 26 | Ni5-Cu1/PP | 250 | 1 | Acetone | Cyclohexanol | 81 | 80 | 92 |
| 27 | Ni5-Cu1/PP | 145 | 1 | Acetone | Ethanolamine | 85 | 86 | 90 |
| 28 | Ni5-Cu1/PP | 175 | 1 | Cyclohexanone | Cyclohexanol | 86 | 81 | 91 |
| 29 | Ni5-Cu1/PE | 175 | 1 | Butanone | Propanol | 82 | 88 | 91 |
| 30 | Ni5-Cu1/PE/ | 210 | 0.5 | Cyclohexanone | Ethanol | 86 | 89 | 93 |
| | ceramic | | | | | | | |

At the same time, it is verified that although different process parameters were used in Examples 11 to 30, the performance of the catalysts prepared therefrom is basically the same, proving that the supported catalyst of the present disclosure could efficiently catalyze the condensation coupling of α-H ketone and alcohol.

In summary, when the catalyst prepared above is used in the condensation coupling of α-H-containing ketone and alcohol, the reaction does not require an external solvent or high-pressure hydrogen. The reaction is easy to be implemented, the catalyst raw materials used are easy to be obtained, and the preparation method is simple. In addition, a catalytic conversion rate for condensation coupling is high and the applicable alcohol categories are wide. The abundant small-molecule alcohol and α-H-containing ketone can be efficiently reacted to obtain the high-carbon ketone, with conversion rates of the alcohol and ketone of not less than 80% respectively, and a selectivity of high-carbon ketone of not less than 90%. In addition, the preparation method can operate stably for a long time and has desirable industrial application prospects.

The embodiments of the present disclosure have been described above. Without conflict, the embodiments and features in the embodiments of the present disclosure can be combined with each other. The present disclosure is not limited to the foregoing specific embodiments, which are only illustrative and not restrictive. Under the inspiration of the present disclosure, those skilled in the art can make many improvements without departing from the purpose of the present disclosure and the scope defined by the claims, and these improvements shall fall within the scope of the present disclosure.

What is claimed is:

1. A method for preparing a supported catalyst, comprising:
mixing a transition metal nitrate and/or a transition metal acetate as a reaction substrate, a polyolefin powder porous material as a catalyst carrier, and water as a reaction medium to obtain a mixture; and
subjecting the mixture to drying and calcination in sequence to obtain the supported catalyst.

2. The method of claim 1, wherein a transition metal in the transition metal nitrate and/or the transition metal acetate is an element selected from the group consisting of Group VIIB, Group VIII, Group IB, and Group IIB in periodic table of elements, and the transition metal is a non-precious metal element.

3. The method of claim 2, wherein the transition metal is selected from a transition metal element in a fourth period of the periodic table of elements.

4. The method of claim 1, comprising the following steps:
S1, dissolving the transition metal nitrate and/or the transition metal acetate in the water to obtain a solution;
S2, adding the polyolefin powder porous material into the solution obtained in step S1 and mixing by stirring at ambient temperature to obtain a mixed system;
S3, placing the mixed system obtained in step S2 in an oven, and drying the mixed system at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

5. The method of claim 2, comprising the following steps:
S1, dissolving the transition metal nitrate and/or the transition metal acetate in an appropriate amount of the water to obtain a solution;
S2, adding the polyolefin powder porous material into the solution obtained in step S1 and mixing evenly by stirring at ambient temperature to obtain a mixed system;
S3, placing the mixed system obtained in step S2 in an oven, and drying the mixed system at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

6. The method of claim 3, comprising the following steps:
S1, dissolving the transition metal nitrate and/or the transition metal acetate in an appropriate amount of the water to obtain a solution;
S2, adding the polyolefin powder porous material into the solution obtained in step S1 and mixing evenly by stirring at ambient temperature to obtain a mixed system;
S3, placing the mixed system obtained in step S2 in an oven, and drying the mixed system at a temperature of 80° C. to 120° C. to a constant weight to obtain a dried material; and
S4, placing the dried material obtained in step S3 in a calcination device, heating and calcinating the dried material at a calcination temperature of 700° C. to 900° C. for 2 h to 8 h to reduce the transition metal in situ during calcination decomposition, and then cooling a resulting material to obtain the supported catalyst.

7. The method of claim 4, wherein the heating is conducted at a heating rate of 2° C./min to 10° C./min, and the calcination temperature is in a range of 750° C. to 850° C.

8. A supported catalyst prepared by the method of claim 1.

9. The supported catalyst of claim 8, wherein the supported catalyst is any one or more selected from the group consisting of Ni5-Cu1/Polypropylene(PP), Ni5/PP, Ni2-Cu1/PP, Ni5-Co1/PP, Ni5-Fe1/PP, Co5-Zn1/PP, Mn5-Cu1/PP, Ni5-Cu1/polyethylene(PE), and Ni5-Cu1/PE.

10. The supported catalyst of claim 8, wherein a transition metal in the transition metal nitrate and/or the transition metal acetate is an element selected from the group consisting of Group VIIB, Group VIII, Group IB, and Group IIB in periodic table of elements, and the transition metal is a non-precious metal element.

11. The supported catalyst of claim 8, wherein the transition metal is selected from a transition metal element in a fourth period of the periodic table of elements.

12. A method for preparing a high-carbon ketone, comprising:
subjecting an alcohol and an α-H-containing ketone that serve as reaction substrates to condensation coupling reaction in the presence of the supported catalyst of claim 8 as a reaction catalyst in a closed reactor at a temperature of 120° C. to 250° C. under an initial pressure of atmospheric pressure to obtain the high-carbon ketone.

13. The method of claim 12, wherein the supported catalyst is any one or more selected from the group consisting of Ni5-Cu1/PP, Ni5/PP, Ni2-Cu1/PP, Ni5-Co1/PP, Ni5-Fe1/PP, Co5-Zn1/PP, Mn5-Cu1/PP, Ni5-Cu1/PE, and Ni5-Cu1/PE.

14. The method of claim 12, wherein a transition metal in the transition metal nitrate and/or the transition metal acetate is an element selected from the group consisting of Group VIIB, Group VIII, Group IB, and Group IIB in periodic table of elements, and the transition metal is a non-precious metal element.

15. The method of claim 12, wherein the transition metal is selected from a transition metal element in a fourth period of the periodic table of elements.

16. The method of claim 12, wherein the alcohol is one or more selected from the group consisting of an aliphatic alcohol, an aromatic alcohol, an alicyclic alcohol, and an alcohol containing other heteroatom substituents; and
the α-H-containing ketone is one or more selected from the group consisting of an aliphatic ketone, an aromatic ketone, an alicyclic ketone, and a ketone containing other heteroatom substituents.

17. The method of claim 12, wherein in the reaction substrates, a molar ratio of the α-H-containing ketone to the alcohol is in a range of 1:2 to 2:1, and a feeding ratio of the α-H-containing ketone to the supported catalyst is 0.2 g to 0.3 g of the supported catalyst per 1 mol of the α-H-containing ketone; and
the condensation coupling reaction is conducted at a temperature of 160° C. to 210° C. for 30 min to 300 min.
